(19) United States
(12) Patent Application Publication
(10) Pub. No.: US 2025/0073088 A1
(43) Pub. Date: Mar. 6, 2025

(54) MULTI-LAYERED ABSORBENT CORES AND ABSORBENT ARTICLES

(71) Applicant: KIMBERLY-CLARK WORLDWIDE, INC., Neenah, WI (US)

(72) Inventors: Lynne KRUEGER, Neenah, WI (US); Carlos J. GARCIA, Neenah, WI (US); Yong Zhao, Neenah, WI (US); Kim DOWNS, Neenah, WI (US)

(21) Appl. No.: 18/816,306

(22) Filed: Aug. 27, 2024

Related U.S. Application Data
(60) Provisional application No. 63/535,376, filed on Aug. 30, 2023.

(51) Int. Cl. A61F 13/15; A61F 13/53; A61F 13/537

(52) U.S. Cl. CPC A61F 13/15203; A61F 13/53708; A61F 2013/15406; A61F 2013/15471; A61F 2013/15512; A61F 2013/530226; A61F 2013/530343; A61F 2013/530489

(57) ABSTRACT

Disclosed herein are multilayer absorbent cores, as well as personal care absorbent articles comprising the multilayer absorbent cores. The multilayer absorbent cores can comprise three or more layers including an intake layer and an absorbent layer. By employing a multilayer structure that includes a relatively low basis weight intake layer and a relatively high basis weight absorbent layer (and thus with a relatively large difference between the basis weight of the intake layer and the basis weight of the absorbent layer), fluid contacting the multilayer absorbent core can be rapidly directed through the intake layer to the absorbent layer. Consequently, articles incorporating these multilayer absorbent cores can exhibit improved intake properties, improved rewet performance, improved saturation and retention capacity, and/or improved desorption.

### CROSS-REFERENCE TO RELATED APPLICATIONS

This application claims benefit of priority of U.S. Provisional Application No. 63/535,376, filed Aug. 30, 2023, which is incorporated herein by reference in its entirety.

### BACKGROUND

Personal care products, such as diapers, diaper pants, training pants, adult incontinence products, and feminine care products, can include absorbent structures that are intended to provide various functional characteristics. For example, absorbent structures in such products are intended to intake body exudates sufficiently quickly, distribute such exudates to an absorbent core or body that is capable of storing an adequate volume of exudates, and prevent such stored exudates in the absorbent core from exiting the absorbent core and transferring to other layers of the product and/or against the user's skin or clothing. Personal care products must also be considerate of other user perceived benefits such as comfort and discreteness, which can be impacted by absorbent structure properties such as thickness (wet thickness and/or dry thickness), stiffness, and weight.

Producing a multilayer absorbent material with satisfactory properties in each of these categories proves difficult because designing an absorbent structure to enhance one property can negatively affect other properties. For example, the storage capacity (saturation capacity) of an absorbent structure can be increased by adding more absorbent fibers or superabsorbent material into an absorbent layer of an absorbent structure, however, such addition of material can increase the thickness (dry and/or wet) of the absorbent material. Further, modifying the absorbent structure to improve the intake properties of the absorbent structure can negatively impact the rewet properties of the absorbent structure.

Thus, there exists a need to develop multi-layer absorbent materials that can provide different functionalities between various layers and that provide necessary intake functionality and rewet properties, while remaining sufficiently thin.

### SUMMARY

Provided herein are multilayer absorbent cores that comprise three or more layers including an intake layer and an absorbent layer. In some implementations, the intake layer can have a relatively low basis weight (e.g., a basis weight of from 70 grams per square meter (gsm) to 120 gsm, or a basis weight of from 80 gsm to 100 gsm) and the absorbent layer can have a relatively high basis weight (e.g., a basis weight of from 200 gsm to 300 gsm, or from 250 gsm to 270 gsm). The ratio of the basis weight of the intake layer to the basis weight of the absorbent layer can be from 0.25 to 0.5 (e.g., from 0.3 to 0.4).

By employing a multilayer structure that includes a relatively low basis weight intake layer and a relatively high basis weight absorbent layer (and thus with a relatively large difference between the basis weight of the intake layer and the basis weight of the absorbent layer), fluid contacting the multilayer absorbent core can be rapidly directed through the intake layer to the absorbent layer. Consequently, articles incorporating these multilayer absorbent cores according to some implementations can exhibit improved intake properties, improved rewet performance, improved saturation and retention capacity, improved desorption, or combinations thereof.

For example, in some implementations, provided herein are multilayer absorbent cores that comprise a transfer layer having a basis weight of from 100 gsm to 175 gsm (e.g., from 120 gsm to 140 gsm); an absorbent layer having a basis weight of from 200 gsm to 300 gsm (e.g., from 250 gsm to 270 gsm); and an intake layer positioned between and in fluid communication with the transfer layer and the absorbent layer, the intake layer having a basis weight of from 70 gsm to 120 gsm (e.g., from 80 gsm to 100 gsm). In some implementations, the ratio of the basis weight of the intake layer to the basis weight of the absorbent layer can be from 0.25 to 0.5 (e.g., from 0.3 to 0.4).

Also provided are personal care absorbent articles for receiving a body exudate. These articles can comprise a liquid-permeable topsheet layer, a liquid-impermeable backsheet layer, and a multilayer absorbent core described herein positioned between the topsheet layer and the backsheet layer.

In some implementations, these articles can exhibit improved intake properties, improved rewet performance, improved saturation and retention capacity, improved desorption, or combinations thereof. In some implementations, the article can exhibit a rewet value of less than 1.0 g, as determined by a Rewet Test, such as a rewet value of from 0.55 g to 0.90 g, or from 0.55 g to 0.75 g. In some implementations, the article can exhibit a retention capacity of at least 35 g/g, as determined by a Retention Capacity Test, such as a retention capacity of from 35 g/g to 42 g/g.

### DESCRIPTION OF DRAWINGS

FIG. 1 is a top perspective view of an example absorbent article in the form of a feminine hygienic pad according to one implementation.

FIG. 2 is a partial exploded perspective view of the example pad illustrated in FIG. 1.

FIG. 3 is a cross-sectional view of a topsheet of the example pad illustrated in FIG. 1, taken along line 1B-1B, according to one implementation.

FIG. 4 is a cross-sectional view of the topsheet of the example pad illustrated in FIG. 1, taken along line 1B-1B, according to another implementation.

FIG. 5 is a cross-sectional view of the topsheet of the example pad illustrated in FIG. 1, taken along line 1B-1B, according to another implementation.

FIG. 6 is a partial top perspective view of a topsheet of the example pad illustrated in FIG. 1, with the topsheet layer removed.

FIG. 7 is a cross-sectional view of an example multilayer absorbent core according to one implementation.

FIG. 8 is a partially cut away top view of a Saturated Capacity tester (also referred to as a SAT CAP Box).

FIG. 9 is a perspective view of an example test apparatus for measuring heated gush intake time.

FIG. 10 is a perspective view of a mounting bracket and intake mold used for measuring heated gush intake time.

FIGS. 11A and 11B are photographs showing the test apparatus for measuring heated gush intake time.

FIG. 11C is a photograph showing the silicone tubing, cannula adaptor, and cannula used to deliver menses simulant during measurement of the heated gush intake time.

FIG. 11D illustrates the alignment of the cannula over the insult orifice during measurement of the heated gush intake time.

### DETAILED DESCRIPTION

Multilayer absorbent cores having a relatively large difference between a basis weight of an intake layer and a basis weight of an absorbent layer can more rapidly direct fluid through the intake layer to the absorbent layer, according to various implementations. Consequently, articles incorporating these multilayer absorbent cores, according to some implementations, can exhibit improved intake properties, improved rewet performance, improved saturation and retention capacity, improved desorption, or combinations thereof.

Various implementations of multilayer absorbent cores comprise three or more layers including an intake layer and an absorbent layer. In some implementations, the intake layer can have a relatively low basis weight (e.g., a basis weight of from 70 grams per square meter (gsm) to 120 gsm, or a basis weight of from 80 gsm to 100 gsm) and the absorbent layer can have a relatively high basis weight (e.g., a basis weight of from 200 gsm to 300 gsm, or from 250 gsm to 270 gsm). The ratio of the basis weight of the intake layer to the basis weight of the absorbent layer can be from 0.25 to 0.5 (e.g., from 0.3 to 0.4).

For example, in some implementations, provided herein are multilayer absorbent cores that comprise a transfer layer having a basis weight of from 100 gsm to 175 gsm (e.g., from 120 gsm to 140 gsm); an absorbent layer having a basis weight of from 200 gsm to 300 gsm (e.g., from 250 gsm to 270 gsm); and an intake layer positioned between and in fluid communication with the transfer layer and the absorbent layer, the intake layer having a basis weight of from 70 gsm to 120 gsm (e.g., from 80 gsm to 100 gsm). In some implementations, the ratio of the basis weight of the intake layer to the basis weight of the absorbent layer can be from 0.25 to 0.5 (e.g., from 0.3 to 0.4).

Also provided herein are personal care absorbent articles for receiving a body exudate. These articles can comprise a liquid-permeable topsheet layer, a liquid-impermeable backsheet layer, and a multilayer absorbent core described herein positioned between the topsheet layer and the backsheet layer.

In some implementations, these articles can exhibit improved intake properties, improved rewet performance, improved saturation and retention capacity, improved desorption, or combinations thereof. In some implementations, the article can exhibit a rewet value of less than 1.0 g, as determined by a Rewet Test, such as a rewet value of from 0.55 g to 0.90 g, or from 0.55 g to 0.75 g. In some implementations, the article can exhibit a retention capacity of at least 35 g/g, as determined by a Retention Capacity Test, such as a retention capacity of from 35 g/g to 42 g/g.

The present disclosure can be understood more readily by reference to the following detailed description, examples, drawings, and claims, and their previous and following description. However, before the present articles, systems, and/or methods are disclosed and described, it is to be understood that this disclosure is not limited to the specific or exemplary aspects of articles, systems, and/or methods disclosed unless otherwise specified, as such can, of course, vary. It is also to be understood that the terminology used herein is for the purpose of describing particular aspects only and is not intended to be limiting.

The following description of the disclosure is provided as an enabling teaching of the disclosure in its best, currently known aspect. To this end, those skilled in the relevant art will recognize and appreciate that many changes can be made to the various aspects of the disclosure described herein while still obtaining the beneficial results of the present disclosure. It will also be apparent that some of the desired benefits of the present disclosure can be obtained by selecting some of the features of the present disclosure without utilizing other features. Accordingly, those of ordinary skill in the pertinent art will recognize that many modifications and adaptations to the present disclosure are possible and may even be desirable in certain circumstances and are a part of the present disclosure. Thus, the following description is again provided as illustrative of the principles of the present disclosure and not in limitation thereof.

### Definitions

Unless otherwise defined, all technical and scientific terms used herein have the same meaning as commonly understood by one of ordinary skill in the field of multi-layered absorbent cores and articles. The materials, methods, and examples are illustrative only and are not intended to be limiting.

The term “absorbent article” refers herein to an article which may be placed against or in proximity to the body (i.e., contiguous with the body) of the wearer to absorb and contain various liquid, solid, and semi-solid exudates discharged from the body. Such absorbent articles, as described herein, are intended to be discarded after a limited period of use instead of being laundered or otherwise restored for reuse. It is to be understood that the present disclosure is applicable to various disposable absorbent articles, including, but not limited to, diapers, training pants, youth pants, swim pants, feminine hygiene products, including, but not limited to, menstrual pads, incontinence products, medical garments, surgical pads and bandages, other personal care or health care garments, and the like without departing from the scope of the present disclosure.

The phrase “absorbent core” refers herein to the component of an absorbent article that intakes and retains a majority of a fluid insult. A “multi-layer absorbent core” refers to an absorbent core that is an absorbent laminate formed from two or more nonwoven layers.

The term “nonwoven” refers herein to materials and webs of material which are formed without the aid of a textile weaving or knitting process. The materials and webs of materials can have a structure of individual fibers, filaments, or threads (collectively referred to as “fibers”) which can be interlaid, but not in an identifiable manner as in a knitted fabric. Nonwoven materials or webs can be formed from many processes such as, but not limited to, meltblowing processes, spunbonding processes, carded web processes, etc. The basis weight of nonwoven webs may generally vary, such as from 5 grams per square meter (“gsm”) to 300 gsm.

The term “meltblown” refers herein to fibers formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameter, which can be a microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al. Meltblown fibers are microfibers, which may be continuous or discontinuous, are generally smaller than 10 microns in diameter, and may be tacky and self-bonding when deposited onto a collecting surface.

The term “spunbond” refers herein to small diameter fibers which are formed by extruding molten thermoplastic material as filaments from a plurality of fine capillaries of a spinnerette having a circular or other configuration, with the diameter of the extruded filaments then being rapidly reduced by a conventional process such as, for example, eductive drawing, and processes that are described in U.S. Pat. No. 4,340,563 to Appel et al., U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Peterson, U.S. Pat. No. 3,542,615 to Dobo et al., and U.S. Pat. No. 5,382,400 to Pike. Spunbond fibers are generally continuous and may sometimes have diameters less than 40 microns, and often from 5 microns to 20 microns. Spunbond fibers are generally not tacky when they are deposited on a collecting surface.

The term “airlaid” is well defined in literature and documented in the art. Examples include the DanWeb process as described in U.S. Pat. No. 4,640,810 Laursen et al. assigned to Scan Web of North America Inc; the Kroyer process as described in U.S. Pat. No. 4,494,278 Kroyer et al. and U.S. Pat. No. 5,527,171 Socrensen assigned to Niro Separation a/s; the method of U.S. Pat. No. 4,375,448 Appel et al. assigned to Kimberly-Clark Corporation, or other similar methods. The webs produced by these methods are subsequently bonded together to form an adequate tensile strength web by thermal fusing, latex bonding or combinations thereof, which are well known in the art.

As used herein, the phrase “bonded carded web” refers to nonwoven webs formed by carding processes as are known to those skilled in the art and further described, for example, in coassigned U.S. Pat. No. 4,488,928 to Alikhan and Schmidt. Briefly, carding processes involve starting with a blend of, for example, staple fibers with bonding fibers or other bonding components in a bulky batt that is combed or otherwise treated to provide a generally uniform basis weight. This web is heated or otherwise treated to activate the adhesive component resulting in an integrated, usually lofty nonwoven layer.

The term “coform” refers herein to a process in which at least one meltblown diehead is arranged near a chute through which other materials are added to the web while it is forming. Such other materials may be pulp, superabsorbent or other particles, natural polymer (for example, rayon or cotton) and/or synthetic polymer (for example, polypropylene, polyester, polyamide or acrylic) fibers, for example, where the fibers may have typical lengths ranging from 3 to 52 millimeters long. Coform processes are shown in commonly assigned U.S. Pat. No. 4,818,464 to Lau and 4,100,324 to Anderson et al. Webs produced by the coform process are generally referred to as coform materials.

The term “bonded” refers herein to the joining, adhering, connecting, attaching, or the like, of two elements. Two elements will be considered bonded together when they are joined, adhered, connected, attached, or the like, directly to one another or indirectly to one another, such as when each is directly bonded to intermediate elements. Bonding of nonwoven webs may be achieved by a number of methods, such as powder bonding, wherein a powdered adhesive is distributed through the web and then activated, usually by heating the web and adhesive with hot air, pattern bonding, wherein heated calendar rolls or ultrasonic bonding equipment are used to bond the fibers together, usually in a localized bond pattern, though the web can be bonded across its entire surface if so desired, through-air bonding, wherein air which is sufficiently hot to soften at least one component of the web is directed through the web, chemical bonding using, for example, latex adhesives which are deposited onto the web by, for example, spraying, and consolidation by mechanical methods such as needling and hydroentanglement

As used herein, the phrase “binding agent” refers to materials which are capable of attaching themselves to a substrate or are capable of attaching other substances to a substrate.

The terms “superabsorbent polymer,” “superabsorbent,” and “SAP” are used herein interchangeably and refer to a water-swellable, water-insoluble organic or inorganic material capable, under the most favorable conditions, of absorbing at least 15 times its weight and, in one implementation, at least 30 times its weight, in an aqueous solution containing 0.9 weight percent sodium chloride. The superabsorbent materials can be natural, synthetic and modified natural polymers and materials. In addition, the superabsorbent materials can be inorganic materials, such as silica gels, or organic compounds, such as cross-linked polymers. In some cases, SAPs are produced by polymerization of acrylic acid blended with sodium hydroxide in the presence of an initiator to form a poly-acrylic acid sodium salt (sometimes referred to as sodium polyacrylate). Other materials are also used to make a superabsorbent polymer, such as polyacrylamide copolymer, ethylene maleic anhydride copolymer, cross-linked carboxymethylcellulose, polyvinyl alcohol copolymers, cross-linked polyethylene oxide, and starch grafted copolymer of polyacrylonitrile. SAPs may be present in absorbent articles in particle or fibrous form.

The term “menses simulant” refers herein to a material that simulates the viscoelastic and other properties of menses. The phrase “menses simulant” describes a liquid generally characterized as being a viscoelastic fluid comprising multiple components having inhomogeneous physical and/or chemical properties. It is the inhomogeneous properties of the multiple components that challenge the efficacy of an absorbent or adsorbent material in the handling of complex liquids. In contrast with complex liquids, simple liquids, such as, for example, urine, physiological saline, water, and the like, are generally characterized as having a relatively low viscosity and comprise one or more components having homogeneous physical and/or chemical properties. As a result of having homogeneous properties, the one or more components of simple liquids behave substantially similarly during absorption or adsorption, although some components may be absorbed or adsorbed more readily than others. Although a complex liquid is generally characterized herein as including specific components having inhomogeneous properties, each specific component of a complex liquid generally has homogeneous properties. Consider for example a representative complex body-liquid having three specific components: red blood cells, blood protein molecules, and water molecules. Upon examination, one skilled in the art could easily distinguish between each of the three specific components according to their generally inhomogeneous properties. Moreover, when examining a particular specific component, such as the red blood cell component, one skilled in the art could easily recognize the generally homogeneous properties of the red blood cells. For example, two menses simulants are used for testing the components described herein. Menses simulant A is defibrinated swine blood at 35% rbc, which is swine blood diluted with swine plasma to provide a hematocrit level of 35-percent (by volume). A suitable device for determining the hematocrit level is a HEMATOSTAT-2 system, available from Separation Technology, Inc., a business having offices located in Altamonte Springs, Fla., U.S.A. A substantially equivalent system may alternatively be employed. Menses simulant B is swine blood diluted to a hematocrit level of 30-percent by volume, with sheared, hick egg white added to mimic the mucin component of menses. Both simulants are available from Cocalico Biologicals, Inc., a business having offices located in Reamstown, Pa., U.S.A.; simulant B is also described in U.S. Pat. No. 5,883,231.

The term “liquid impermeable” refers herein to a layer or multi-layer laminate in which liquid body exudates, such as urine, will not pass through the layer or laminate, under ordinary use conditions, in a direction generally perpendicular to the plane of the layer or laminate at the point of liquid contact.

The term “liquid permeable” refers herein to any material that is not liquid impermeable.

The term “intake” refers herein to the ability of an absorbent article to absorb fluid. Intake time is used to assess the quality of absorption with lower intake times denoting materials capable of rapid absorption and higher intake times denoting materials with poorer absorption.

As used in this application and in the claims, the singular forms “a,” “an,” and “the” include the plural forms unless the context clearly dictates otherwise.

As used in the specification and in the claims, the term “comprising” can include the aspects “consisting of” and “consisting essentially of.” Additionally, the term “includes” means “comprises.”

For the terms “for example,” “exemplary,” and “such as,” and grammatical equivalences thereof, the phrase “and without limitation” is understood to follow unless explicitly stated otherwise.

Ranges can be expressed herein as from one particular value to another particular value. When such a range is expressed, the range includes the end points of the range. It should be further understood that the endpoints of each of the ranges are significant both in relation to the other endpoint and independently of the other endpoint. The description in range format is merely for convenience and brevity and should not be construed as an inflexible limitation on the scope of the disclosure. Accordingly, the description of a range should be considered to have specifically disclosed all the possible subranges as well as individual numerical values within that range. For example, a description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6 and any whole and partial increments therebetween. This applies regardless of the breadth of the range.

As used herein, the terms “optional” or “optionally” mean that the subsequently described event or circumstance may or may not occur and that the description includes instances where said event or circumstance occurs and instances where it does not.

Further, the terms “coupled” and “associated” generally mean electrically, electromagnetically, and/or physically (e.g., mechanically or chemically) coupled or linked and do not exclude the presence of intermediate elements between the coupled or associated items.

As used herein, the term “and/or” includes any and all combinations of one or more of the associated listed items.

It will be understood that the terms “first,” “second,” etc., may be used herein to describe various elements, components, regions, layers, and/or sections. These elements, components, regions, layers, and/or sections should not be limited by these terms. These terms are only used to distinguish one element, component, region, layer, or section from another element, component, region, layer, or a section. Thus, a first element, component, region, layer, or section discussed below could be termed a second element, component, region, layer, or section without departing from the teachings of exemplary aspects.

Spatially relative terms, such as “beneath,” “below,” “lower,” “above,” “upper,” and the like, may be used herein for ease of description to describe one element or feature's relationship to another element(s) or feature(s). It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” can encompass both an orientation of above and below. The device may be otherwise oriented (rotated 90 degrees or at other orientations), and the spatially relative descriptors used herein are interpreted accordingly.

As used herein, the term “substantially” means that the subsequently described event or circumstance completely occurs or that the subsequently described event or circumstance generally, typically, or approximately occurs. Still further, the term “substantially” can, in some aspects, refer to at least 80%, at least 85%, at least 90%, at least 91%, at least 92%, at least 93%, at least 94%, at least 95%, at least 96%, at least 97%, at least 98%, at least 99%, or 100% of the stated property, component, composition, or other condition for which substantially is used to characterize or otherwise quantify an amount.

Reference now will be made in detail to various implementations, one or more examples of which are set forth below. Each example is provided by way of explanation, not as a limitation. In fact, it will be apparent to those skilled in the art that various modifications and variations may be made from this disclosure without departing from the scope or spirit of the claims. For instance, features illustrated or described as part of one implementation, may be used on another implementation to yield a still further implementation. For the purposes of this application, like features may be represented by like numbers between the figures.

While not illustrated in most figures except where additional location emphasis is desired, it should be understood that traditional absorbent article construction adhesive (or other bonding technology) is to be used to fasten the various layers of the described articles together. Such construction adhesive or other bonding technology can be placed so as not to interfere with the flow of fluid/liquid waste through the article, such as in the depth direction towards the multilayer absorbent core, or in a location so as not to interfere with or impede the occurrence of desired spatial gaps or void spaces as noted. For example, adhesive or other bonding techniques are desirably used only between or at the peripheral side edges of immediately subjacent layers so as to avoid interfering with liquid flow and to preserve spatial gaps (thereby avoiding unnecessary subjacent layer contact in specific regions). In noted absorbent article areas where void spaces are potentially present, adhesive or other bonding features would be desirably absent or present in limited amounts.

In some examples, the personal care absorbent articles described herein are ideally suitable for use as hygiene articles in the feminine and adult care product categories. Such articles include for example, feminine hygiene sanitary pads and liners, and adult care garment inserts, pads, and liners. While pads are illustrated in the figures, it should be understood however, that absorbent articles are not meant to be limited to such product applications. For instance, baby and child care product applications are similarly contemplated to be within the scope of the term “absorbent articles.” In addition, while the primary body exudate utilized in the description is menses, other body exudates are contemplated including, but not limited to, blood, feces, urine and other body fluids.

### Multilayer Absorbent Cores

Provided herein are multilayer absorbent cores that comprise three or more layers including an intake layer and an absorbent layer. As discussed in more detail below, the intake layer can have a relatively low basis weight (e.g., a basis weight of from 70 gsm to 120 gsm, or a basis weight of from 80 gsm to 100 gsm) and the absorbent layer can have a relatively high basis weight (e.g., a basis weight of from 200 gsm to 300 gsm, or from 250 gsm to 270 gsm). The ratio of the basis weight of the intake layer to the basis weight of the absorbent layer can be from 0.25 to 0.5 (e.g., from 0.3 to 0.4).

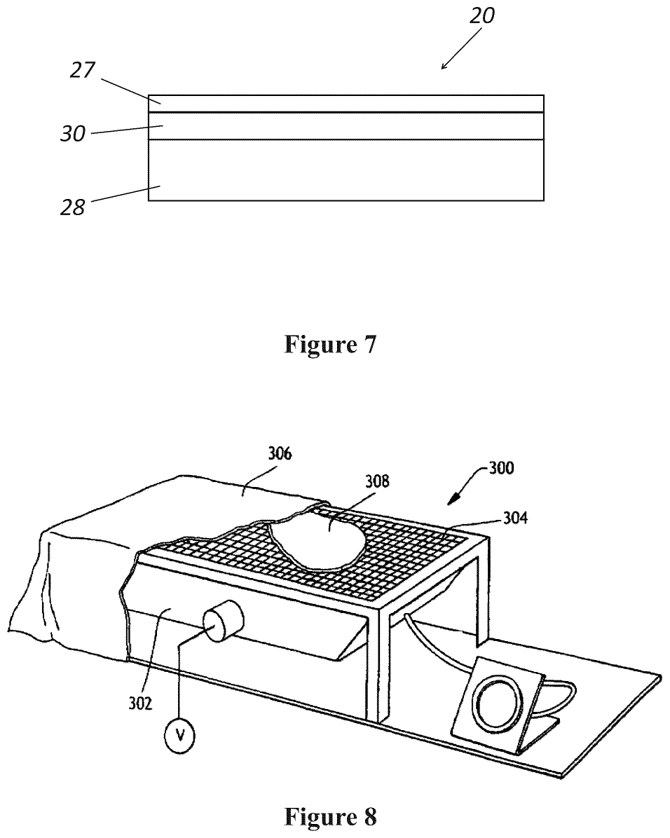

In some of these implementations, the multilayer absorbent core can further comprise a transfer layer having a basis weight that is greater than the basis weight of the intake layer and less than the basis weight of the absorbent layer (e.g., a basis weight of from 100 gsm to 175 gsm, or a basis weight of from 120 gsm to 140 gsm). In the implementations having a transfer layer, the intake layer can be disposed between the transfer layer and the absorbent layer. The transfer layer is oriented within an absorbent article to be closer to the body of the wearer in use, and the absorbent layer is oriented to be furthest from the body of the wearer in use. For example, FIG. 7 shows on implementation of the multilayer absorbent core 20 that comprises a transfer layer 27, an absorbent layer 28, and an intake layer 30 positioned between and in fluid communication with the transfer layer 27 and the absorbent layer 28.

By employing a multilayer structure that includes a relatively low basis weight intake layer and a relatively high basis weight absorbent layer (and thus with a relatively large difference between the basis weight of the intake layer and the basis weight of the absorbent layer), fluid contacting the multilayer absorbent core can be rapidly directed through the intake layer to the absorbent layer. Consequently, articles incorporating these multilayer absorbent cores can exhibit improved intake properties, improved rewet performance, improved saturation and retention capacity, improved desorption, or combinations thereof.

The dimensions of the multilayer absorbent core (as well as the constituent layers) can be varied depending on the demands for an intended application for the multilayer absorbent core. For example, in some implementations, the multilayer absorbent core can provide capacity to absorb and retain bodily exudates. More particularly, the multilayer absorbent core can be configured to hold a liquid, such as urine, menses, complex liquid or the like, as well as combinations thereof.

In some implementations, multilayer absorbent core (and the composition of the layers making up the multilayer absorbent core) can be selected such that the multilayer absorbent core demonstrates a particular total absorbency capacity, depending on the article type. For example, for feminine care products, the total absorbency capacity can typically be within the range of 7 to 50 grams of menstrual fluid, and can more typically be within the range of 30 to 40 g of menstrual fluid. Within the feminine care hygienic article category, it may be desirable to have different levels of absorbency capacity depending on product type. For example, feminine care panty liners are typically used by consumers for “light” menstrual flow days, feminine care pads are typically used by consumers for “regular” menstrual flow days, and feminine care oversized pads are typically used by consumers for “overnight” timespans, or “heavy” menstrual flow days. It may be desirable for feminine care liners to have in one implementation, an absorbency capacity of 1 to 5 grams of fluid. For feminine care pads, it may be desirable in one implementation, to have an absorbency capacity of 10 to 30 grams of fluid. For feminine care oversized pads, in one implementation, it may be desirable to have an absorbency capacity of 20 to 50 grams of fluid.

In some implementations, the multilayer absorbent core can demonstrate some level of compressibility, conformability, can be non-irritating to a wearer's skin, and/or can be capable of absorbing and retaining liquids and certain other body wastes. For example, the multilayer absorbent core can include layers formed from components including cellulosic fibers (e.g., wood pulp fibers), other natural fibers, synthetic fibers, woven or nonwoven sheets, scrim netting or other stabilizing structures, superabsorbent materials, binder materials, surfactants, selected hydrophobic and hydrophilic materials, pigments, lotions, odor control agents or the like, decolorization agents, as well as combinations thereof.

In some implementations, the transfer layer can have a basis weight of at least 100 gsm (e.g., at least 105 gsm, at least 110 gsm, at least 115 gsm, at least 120 gsm, at least 125 gsm, at least 130 gsm, at least 135 gsm, at least 140 gsm, at least 145 gsm, at least 150 gsm, at least 155 gsm, at least 160 gsm, at least 165 gsm, or at least 170 gsm). In some implementations, the transfer layer can have a basis weight of 175 gsm or less (e.g., 170 gsm or less, 165 gsm or less, 160 gsm or less, 155 gsm or less, 150 gsm or less, 145 gsm or less, 140 gsm or less, 135 gsm or less, 130 gsm or less, 125 gsm or less, 120 gsm or less, 115 gsm or less, 110 gsm or less, or 105 gsm or less).

The transfer layer can have a basis weight ranging from any of the minimum values described above to any of the maximum values described above. For example, in some implementations, the transfer layer can have a basis weight of from 100 gsm to 175 gsm (e.g., from 110 gsm to 150 gsm, or from 120 gsm to 140 gsm). In certain implementations, the transfer layer can have a basis weight of 115 gsm to 140 gsm.

In some implementations, the intake layer can have a basis weight of at least 70 gsm (e.g., at least 75 gsm, at least 80 gsm, at least 85 gsm, at least 90 gsm, at least 95 gsm, at least 100 gsm, at least 105 gsm, at least 110 gsm, or at least 115 gsm). In some implementations, the intake layer can have a basis weight of 120 gsm or less (e.g., 115 gsm or less, 110 gsm or less, 105 gsm or less, 100 gsm or less, 95 gsm or less, 90 gsm or less, 85 gsm or less, 80 gsm or less, or 75 gsm or less).

The intake layer can have a basis weight ranging from any of the minimum values described above to any of the maximum values described above. For example, in some implementations, the intake layer can have a basis weight of from 70 gsm to 120 gsm (e.g., from 75 gsm to 110 gsm, or from 80 gsm to 100 gsm). In certain implementations, the intake layer can have a basis weight of from 78 gsm to 102 gsm.

In some implementations, the absorbent layer can have a basis weight of at least 200 gsm (e.g., at least 205 gsm, at least 210 gsm, at least 215 gsm, at least 220 gsm, at least 225 gsm, at least 230 gsm, at least 235 gsm, at least 240 gsm, at least 245 gsm, at least 250 gsm, at least 255 gsm, at least 260 gsm, at least 265 gsm, at least 270 gsm, at least 275 gsm, at least 280 gsm, at least 285 gsm, at least 290 gsm, or at least 295 gsm). In some implementations, the absorbent layer can have a basis weight of 300 gsm or less (e.g., 295 gsm or less, 290 gsm or less, 285 gsm or less, 280 gsm or less, 275 gsm or less, 270 gsm or less, 265 gsm or less, 260 gsm or less, 255 gsm or less, 250 gsm or less, 245 gsm or less, 240 gsm or less, 235 gsm or less, 230 gsm or less, 225 gsm or less, 220 gsm or less, 215 gsm or less, 210 gsm or less, or 205 gsm or less).

The absorbent layer can have a basis weight ranging from any of the minimum values described above to any of the maximum values described above. For example, in some implementations, the absorbent layer can have a basis weight of from 200 gsm to 300 gsm (e.g., from 220 gsm to 290 gsm, from 230 gsm to 290 gsm, from 240 gsm to 280 gsm, or from 250 gsm to 270 gsm). In certain implementations, the absorbent layer can have a basis weight of from 237 gsm to 283 gsm.

The basis weight of the intake layer and the basis weight of the absorbent can be selected in combination so as to drive fluid transfer from the intake layer to the absorbent layer. By employing an intake layer with a relatively low basis weight and an absorbent layer with a relatively high basis weight, fluid transfer to the absorbent layer can be enhanced (e.g., decreasing intake time).

For example, in some implementations, the ratio of the basis weight of the intake layer to the basis weight of the absorbent layer can be at least 0.25 (e.g., at least 0.3, at least 0.35, at least 0.4, or at least 0.45). In some implementations, the ratio of the basis weight of the intake layer to the basis weight of the absorbent layer can be 0.5 or less (e.g., 0.45 or less, 0.4 or less, 0.35 or less, or 0.3 or less).

The ratio of the basis weight of the intake layer to the basis weight of the absorbent layer ranging from any of the minimum values described above to any of the maximum values described above. For example, in some implementations, the ratio of the basis weight of the intake layer to the basis weight of the absorbent layer can be from 0.25 to 0.5 (e.g., from 0.25 to 0.45, or from 0.3 to 0.4). In certain implementations, the ratio of the basis weight of the intake layer to the basis weight of the absorbent layer can be from 0.28 to 0.43.

The transfer layer can be pliable, less hydrophilic than the absorbent layer, and sufficiently porous to thereby permit liquid body exudates to penetrate through the transfer layer to ultimately reach the absorbent layer. In some implementations, the transfer layer can have sufficient structural integrity to withstand wetting thereof and of the absorbent layer. In some implementations, the transfer layer can be constructed from a single layer of material, or it can be a laminate constructed from two or more layers of material. Further, the hydrophobicity of the transfer layer (relative to the intake layer and/or the absorbent layer) can decrease the likelihood of liquid rewet or flowback to the wearer's skin. If desired, the transfer layer can be perforated to adjust the fluid transfer characteristics of the transfer layer.

In some implementations, the transfer layer can include, but is not limited to, natural and synthetic fibers such as, but not limited to, polyester, polypropylene, acetate, nylon, polymeric materials, cellulosic materials such as wood pulp, cotton, rayon, viscose, LYOCELL® such as from Lenzing Company of Austria, or mixtures of these or other cellulosic fibers, and combinations thereof. Natural fibers can include, but are not limited to, wool, cotton, flax, hemp, and wood pulp. Wood pulps can include, but are not limited to, standard softwood fluffing grade such as “CoosAbsorb™ S Fluff Pulp” or equivalent available from Abitibi Bowater, Greenville, S.C., U.S.A., which is a bleached, highly absorbent sulfate wood pulp containing primarily southern soft wood fibers.

In some implementations, the transfer layer can be formed of a material that is substantially hydrophobic, such as a nonwoven web composed of polypropylene, polyethylene, polyester, etc. One example of a material suitable for the transfer layer is a spunbond web composed of polypropylene, multi-lobal fibers. Further examples of suitable transfer layer materials include spunbond webs composed of polypropylene fibers, which may be round, tri-lobal or poly-lobal in cross-sectional shape and which may be hollow or solid in structure. These webs can be bonded, such as by thermal bonding, over 3% to 30% of the web area. Other examples of suitable materials that may be used for the transfer layer are described in U.S. Pat. No. 4,798,603 to Meyer, et al. and 5,248,309 to Serbiak, et al. To adjust performance, the transfer delay layer may also be treated with a selected amount of wettability additive, such as a surfactant, to increase its initial wettability.

In some implementations, the transfer layer can comprise a laminated multicomponent nonwoven web. In some implementations, the laminated multicomponent nonwoven web can comprise cellulosic fibers.

The intake layer can be liquid-permeable and positioned between the transfer layer and the absorbent layer. The intake layer may be made of a material that is capable of rapidly transferring a body fluid that is delivered to the multilayer absorbent core through the intake layer.

Any of a variety of different materials are capable of being used for the intake layer to accomplish the above-mentioned functions. In some implementations, the intake layer can be formed from one or more materials, such that the intake layer can wick menstrual fluid a distance of 1.2 cm to 15.25 cm (0.5 to 6 inches) in one hour when one end of the material is placed in an infinite reservoir of menses simulant.

In various implementations, the intake layer can be formed of two or more layers of nonwoven material bonded together. Any of a variety of nonwoven materials can be used for the fluid intake layer. For example, suitable nonwoven materials may be synthetic, cellulosic, or a combination of synthetic and cellulosic materials. For example, each layer of the intake layer can be constructed utilizing an airlaid, spunbond, tissue, meltblown, spunbond-meltblown-spunbond, or TABCW material. In some implementations, at least two of the layers of nonwoven material forming the intake layer can be different from each other. For example, in some implementations, the intake layer can comprise a first layer formed from a TABCW material and a second layer formed from an airlaid cellulosic material. In such an example, the two layers can be bonded together to form the intake layer. In such an example, the airlaid cellulosic material can comprise hardwood and/or softwood fibers. An airlaid cellulosic layer can have a fine pore structure and can provide an excellent wicking capacity, especially for menses.

In some implementations, the intake layer can comprise an airlaid nonwoven web. In some implementations, the airlaid nonwoven web can comprise a blend of cellulosic fibers and binder fibers formed from a synthetic polymer. In some implementations, the airlaid nonwoven web is thermal bonded.

The absorbent layer can be formed from a matrix of absorbent fibers and/or absorbent particulate material. The absorbent fibers can include natural and/or synthetic fibers.

In some examples, the absorbent layer can comprise cellulosic fibers (e.g., wood pulp fibers), other natural fibers, synthetic fibers, woven or nonwoven sheets, scrim netting or other stabilizing structures, superabsorbent materials, binder materials, surfactants, selected hydrophobic and hydrophilic materials, pigments, lotions, odor control agents or the like, decolorization agents, as well as combinations thereof. By way of example, suitable materials and/or structures for the absorbent core can include, but are not limited to, those described in U.S. Pat. No. 4,610,678 to Weisman, et al., U.S. Pat. No. 6,060,636 to Yahiaoui, et al., U.S. Pat. No. 6,610,903 to Latimer, et al., U.S. Pat. No. 7,358,282 to Krueger, et al., and U.S. Publication No. 2010/0174260 to Di Luccio, et al.

In some examples, the absorbent layer can be suitably composed of hydrophilic fibers (e.g., cellulosic fluff, such as wood pulp fluff), a superabsorbent material, or a mixture of cellulosic fluff and superabsorbent material. In some examples, the wearer facing portion of the absorbent layer can have a lower absorbent capacity per unit weight than the garment facing portion of the absorbent layer. For example, the wearer facing portion of the absorbent layer may be composed of a mixture of hydrophilic fibers and superabsorbent material, and the concentration of superabsorbent material present in the wearer facing portion of the absorbent layer can be lower than the concentration of superabsorbent material present in the garment facing portion of the absorbent layer so that the wearer facing portion of the absorbent layer can have a lower absorbent capacity per unit weight than the garment facing portion of the absorbent layer. It is also contemplated that the garment facing portion of the absorbent layer may be composed solely of superabsorbent material, or that the wearer facing portion of the absorbent layer may be composed solely of hydrophilic fibers. It is also contemplated that, in some implementations, each of the layers, the wearer facing and garment facing portions of the absorbent layer, can have a superabsorbent material such that the absorbent capacities of the two superabsorbent materials can be different and can provide the absorbent layer with a lower absorbent capacity in the wearer facing portion of the absorbent layer than in the garment facing portion of the absorbent layer.

Various types of wettable, hydrophilic fibers can be used in the absorbent layer. Examples of suitable fibers include natural fibers, cellulosic fibers, synthetic fibers composed of cellulose or cellulose derivatives, such as rayon fibers; inorganic fibers composed of an inherently wettable material, such as glass fibers; synthetic fibers made from inherently wettable thermoplastic polymers, such as particular polyester or polyamide fibers, or composed of nonwettable thermoplastic polymers, such as polyolefin fibers which have been hydrophilized by suitable means. The fibers may be hydrophilized, for example, by treatment with a surfactant, treatment with silica, treatment with a material which has a suitable hydrophilic moiety and is not readily removed from the fiber, or by sheathing the nonwettable, hydrophobic fiber with a hydrophilic polymer during or after formation of the fiber. For example, one suitable type of fiber is a wood pulp that is a bleached, highly absorbent sulfate wood pulp containing primarily soft wood fibers. However, the wood pulp can be exchanged with other fiber materials, such as synthetic, polymeric, or meltblown fibers or with a combination of meltblown and natural fibers. In some implementations, the cellulosic fluff can include a blend of wood pulp fluff. An example of wood pulp fluff can be “CoosAbsorb™ S Fluff Pulp” or equivalent available from Abitibi Bowater, Greenville, S.C., U.S.A., which is a bleached, highly absorbent sulfate wood pulp containing primarily southern soft wood fibers.

In some implementations, the absorbent layer can comprise an airlaid nonwoven web.

In some implementations, the absorbent layer can comprise cellulosic fibers.

In some implementations, the absorbent layer can comprise a web material that includes a matrix of cellulosic fluff, and optionally superabsorbent material.

In some implementations, the absorbent layer can comprise from 5% by weight to 50% by weight of a superabsorbent polymer composition.

If desired, the absorbent layer can include an optional amount of a superabsorbent material. Suitable superabsorbent materials can be selected from natural, synthetic, and modified natural polymers and materials. The superabsorbent materials can be inorganic materials, such as silica gels, or organic compounds, such as cross-linked polymers. Cross-linking may be covalent, ionic, Van der Waals, or hydrogen bonding. In some implementations, a superabsorbent material can be capable of absorbing at least ten times its weight in liquid. In some implementations, the superabsorbent material can absorb more than twenty-four times its weight in liquid. Examples of superabsorbent materials include polyacrylamides, polyvinyl alcohol, ethylene maleic anhydride copolymers, polyvinyl ethers, hydroxypropyl cellulose, carboxymal methyl cellulose, polyvinylmorpholinone, polymers and copolymers of vinyl sulfonic acid, polyacrylates, polyacrylamides, polyvinyl pyrrolidone, and the like. Additional polymers suitable for superabsorbent material include hydrolyzed, acrylonitrile grafted starch, acrylic acid grafted starch, polyacrylates and isobutylene maleic anhydride copolymers and mixtures thereof. The superabsorbent material may be in the form of discrete particles. The discrete particles can be of any desired shape, for example, spiral or semi-spiral, cubic, rod-like, polyhedral, etc. Shapes having a largest greatest dimension/smallest dimension ratio, such as needles, flakes, and fibers are also contemplated for use herein. Conglomerates of particles of superabsorbent materials may also be used.

Regardless of the combination of absorbent materials present in the absorbent layer, the absorbent materials may be formed into a web structure by employing various conventional methods and techniques. For example, the absorbent layer may be formed with a dry-forming technique, an air forming technique, a wet-forming technique, a foam-forming technique, or the like, as well as combinations thereof. A coform nonwoven material may also be employed. In certain implementations, the absorbent layer can comprise an airlaid layer. Methods and apparatus for carrying out such techniques are well known in the art.

### Absorbent Articles

The multilayer absorbent cores described herein can be utilized as absorbent components in absorbent articles. Accordingly, also provided are absorbent articles, including personal care absorbent articles, that comprise the multilayer absorbent cores described herein. While illustrated and discussed below with respect to one example article (a feminine hygienic pad or menstrual pad), it will be understood that the article can comprise any suitable absorbent article, such as for example a diaper, a training pant, a youth pant, a swim pant, a feminine hygiene product (e.g., a menstrual pad or menstrual pant), an incontinence products, a medical garment, a surgical pad, or a bandage.

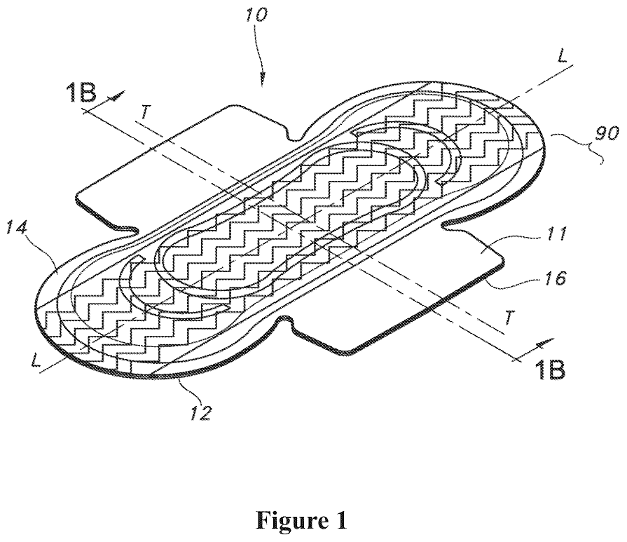

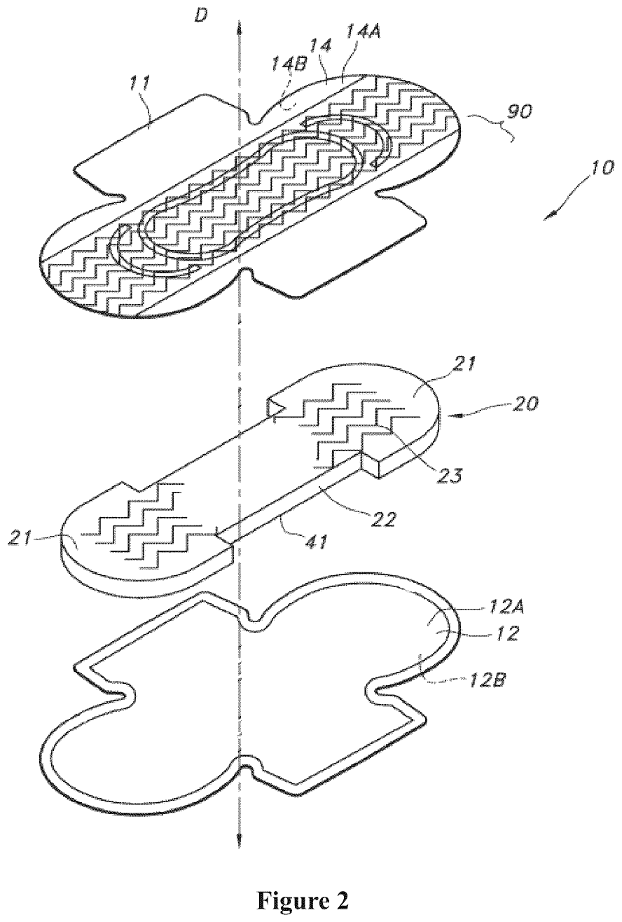

By way of example, FIGS. 1 and 2 illustrate a top perspective and exploded perspective view respectively, of a first implementation of a personal care absorbent article in the form of a feminine hygienic pad (also referred to as a feminine hygiene absorbent personal care article). The pad has a longitudinal axis (including the central longitudinal axis L shown, as indicated by a broken line), a transverse axis (and a central transverse axis T shown) and a vertical or depth or Z-directional axis D (as seen in FIG. 2), which is the direction normal to the plane of the pad layers. The feminine hygienic pad (or feminine hygienic absorbent article) 10 can be shaped in a variety of geometries to facilitate wear by a user. In some implementations, the pad 10 can include side wings 11 extending out at the longitudinally directed side edges 90 of the article.

The pad can comprise a liquid impermeable garment-facing backsheet layer 12 and a liquid permeable, user facing top layer (e.g., topsheet) 14. The backsheet layer 12 and topsheet layer 14 can sandwich a multilayer absorbent core 20, such as those described above. While not expressly labeled, the topsheet layer 14 can optionally include (as shown in FIG. 1) an embossing pattern, such as a wave/zig-zag, racetrack, and/or arc pattern.

The backsheet layer 12, being generally liquid-impermeable, can be designed to face the inner surface, i.e., the crotch portion, of a user's undergarment (not shown) or outer garment. The backsheet therefore includes a core facing surface 12A and an undergarment facing surface 12B. The backsheet layer 12 may optionally permit the passage of air or vapor out of the absorbent article 10, while still blocking the passage of liquids.

A variety of suitable liquid-impermeable materials may be utilized to form the backsheet layer 12. For example, in some implementations, the backsheet layer can comprise a microporous polymeric film, such as a polyolefin film of polyethylene or polypropylene. In some examples, a polyethylene film can be utilized having a thickness of from 0.2 mils to 5.0 mils (e.g., from 0.5 to 3.0 mils). In some implementations, the backsheet layer can comprise a calcium carbonate-filled polypropylene film. In some implementations, the backsheet layer can comprise an embossed polyethylene film having a basis weight of from 21 gsm to 27 gsm. In some implementations, the polymeric films can be corona treated. Suitable backsheet layer materials include polyolefin films commercially available from Pliant Plastics Corporation, Schaumburg, III., USA.

In some implementations, the backsheet can comprise a hydrophobic nonwoven material with water barrier properties, such as a nonwoven laminate. For example, the backsheet layer can comprise a four-layered laminate (e.g., a spunbond, meltblown, meltblown, spunbond laminate). The backsheet layer 12 can therefore be of a single layer or multilayer construction, such as of multiple film layers or laminates of film and nonwoven fibrous layers. Even with a film backsheet, a nonwoven fibrous layer may be used as the undergarment facing surface for better “hand” or feel.

In some implementations, a garment attachment adhesive can be applied to the garment-facing side of the backsheet and/or to the garment-facing side of the side wings for fastening the pad directly to the crotch portion of an undergarment. The garment attachment adhesive can be composed of any suitable adhesive. For example, the garment attachment adhesive can be a pressure-sensitive adhesive such as EASYMELT 34-5602, available from National Starch and Chemical Company. If desired, a peel strip can be added to cover the garment attachment adhesive to prevent adhesive contamination. Examples of suitable peel strips include silicone coated Kraft paper, silicone coated film, and the like. Other release coatings include coatings containing polytetrafluoroethylene.

In some implementations, the topsheet layer 14 can be dimensioned so as to surround the multilayer absorbent core 20. For example, the topsheet layer 14 can completely encase the multilayer absorbent core and/or backsheet layer (encasement not shown). Alternatively, the topsheet layer 14 and the backsheet layer 12 may both extend beyond the multilayer absorbent core's lateral-most edges (41, for example) and be peripherally joined together, either entirely or partially, using known attachment techniques. For example, the topsheet layer 14 and the backsheet layer 12 can be joined by adhesive bonding, ultrasonic bonding, or any other suitable joining method known in the art, the sealed edges defining an overall sealed peripheral edge 16 of the feminine hygienic pad 10. The feminine hygienic pad 10 can take on various geometries but will generally have opposite lateral sides (in the product longitudinal direction) and longitudinal ends.

The topsheet layer 14 can be liquid permeable and designed to contact the body of the user. The liquid permeable topsheet layer 14 can have an outwardly user-facing surface 14A that may directly contact the body of the wearer and receive bodily exudates, and an absorbent core-facing surface 14B. The topsheet layer 14 can selected for comfort and conformability, and through its structure can function to direct bodily exudates away from the body of a user and towards the multilayer absorbent core 20. In some examples, the topsheet layer 14 can retains little or no liquid in its structure, so as to provide a relatively comfortable and non-irritating surface next to the tissues within the vestibule of a female wearer.

The topsheet layer 14 can be constructed of any woven, nonwoven or sheet material which is easily penetrated by bodily exudates which contact the surface of the backsheet layer 12. Examples of suitable topsheet materials include natural fiber webs (such as cotton), rayon, bonded carded webs of polyester, polypropylene, polyethylene, nylon, or other heat-bondable fibers, polyolefins, such as copolymers of polypropylene and polyethylene, linear low-density polyethylene, and aliphatic esters such as polylactic acid. Finely perforated films and net materials can also be used, as can laminates of/or combinations of these materials.

A specific example of a suitable topsheet material is a bonded carded web made of polypropylene and polyethylene, such as that obtainable from Sandler Corporation, Germany. Another example of a suitable topsheet material is a titanium oxide-filled embossed polypropylene spunbond having a basis weight of from 16 gsm to 20 gsm, such as that obtainable from Fitesa Simpsonville Inc., United States. Another example of a suitable topsheet material is a two-layer film with a basis weight of from 21 gsm to 27 gsm and comprising a perforated film laminated to a spunbond layer, such as that obtainable from Fitesa Simpsonville Inc., United States. Another example of a suitable topsheet material is a two-layer through air bonded carded web with a basis weight of from 20 gsm to 28 gsm and comprising a polyethylene/polypropylene top layer and a polyethylene/polyethylene terephthalate, such as that obtainable from Xiamen YanJan New Material Co, China. Other suitable topsheet materials include those described in U.S. Pat. No. 4,801,494 to Datta, et al., and U.S. Pat. No. 4,908,026 to Sukiennik, et al.

In some implementations, the topsheet layer 14 can include a plurality of apertures (not shown) formed therethrough to permit body fluid to pass more readily into the multilayer absorbent core 20. The apertures may be randomly or uniformly arranged throughout the topsheet layer 14, or they may be located only in a narrow longitudinal band or strip arranged along the longitudinal axis L of the feminine hygienic pad 10, such as down the central longitudinal axis of the article. The size, shape, diameter and number of apertures may be varied to suit an article's particular needs.

As previously noted, the topsheet layer 14 can be optionally embossed with any desired embossing pattern to define embossed channels. Embossing techniques are well known to those skilled in the art. An embossing pattern not only creates an aesthetically pleasing surface, but the channels may also facilitate intake of menses fluid. Menses fluid will tend to flow along the densified edges of the channels rather than pool on contact points of the topsheet layer 14. The topsheet layer itself may also be formed from one or more layers in a side-by-side arrangement along the longitudinal axis, as described in more detail below. In some implementations, the topsheet layer can have a basis weight of from 15 gsm to 100 gsm.

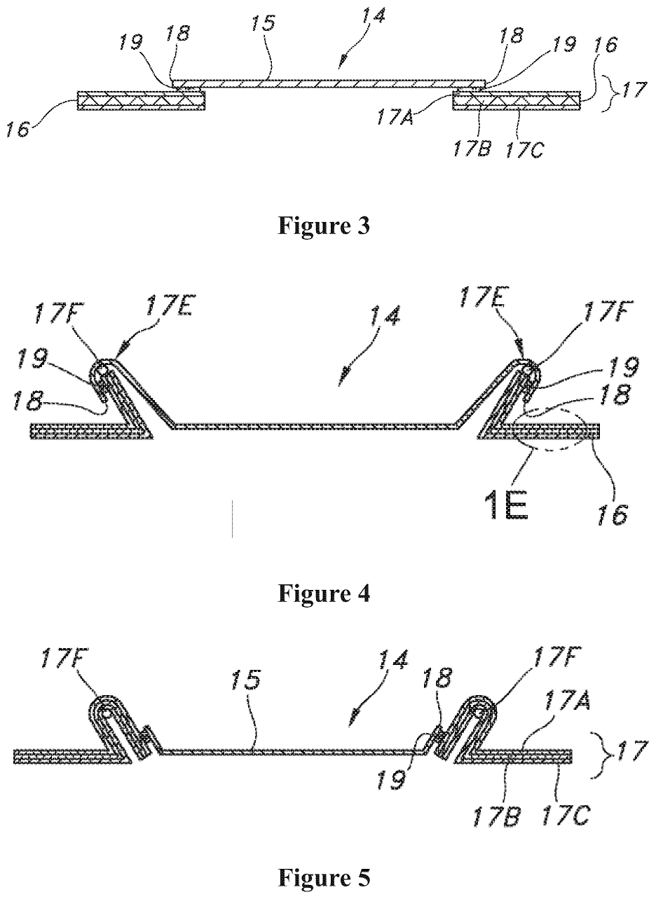

In some implementations, as seen in the cross-sectional view of the topsheet layer 14 of FIG. 3, taken along line 1B-1B of FIG. 1, the topsheet layer 14 can be constructed of at least two different materials 15, 17 in an overlapping, but substantially side-by-side arrangement along the longitudinal axis. Such a dual-layer construction is generally described in U.S. Pat. No. 5,961,505 to Coc, U.S. Pat. No. 5,415,640 to Kirby and 6,117,523 to Sugahara.

By way of example, referring now to FIG. 3, in some implementations, a central longitudinally directed topsheet material 15 can be positioned along the central longitudinal axis L of the topsheet layer 14. In some implementations, the central longitudinally directed topsheet material 15 can be, for example, a through air bonded carded web material (TABCWs) having a basis weight of 15 to 100 gsm. Other nonwoven, woven, and film topsheet materials can also be used as the central longitudinally directed topsheet material 15 of the topsheet layer 14. In some examples, the central longitudinal material can be constructed from a TABCW having a basis weight of from 20 to 50 gsm. Such materials are commercially available from nonwoven material manufacturers, such as Xiamen Yanjan Industry, Beijing Da Yuan Nonwoven Fabrics, and others. Different nonwoven, woven or film sheet materials can be used as the longitudinally directed side edge topsheet materials 17, adjacent and bonded to the central longitudinally directed topsheet material 15.

The topsheet layer 14 materials can be varied based on the overall desired attributes of the topsheet. For example, in some implementations, it can be desired to have a hydrophilic material along the central longitudinal axis and hydrophobic barrier-type materials along the longitudinal side edges to prevent leakage and increase a sensation of dryness at those longitudinal side edges. Such longitudinally directed side edge materials 17 can be either adhesively, thermally, ultrasonically or otherwise bonded 19 to the central longitudinally directed material topsheet 15 along the longitudinally directed side edges 18 (on the absorbent core-facing surface 14B) of the central longitudinally directed topsheet material 15. Such longitudinally directed side edge topsheet materials 17 can be of a single or multiple-layered construction.

In some implementations, the longitudinally directed side edge topsheet materials 17 are themselves adhesively bonded laminates. In some implementations, for example, such longitudinally directed side edge topsheet materials 17, can be constructed of an upper nonwoven layer 17A, such as a meltblown microfiber material (MBMF as further described), one or more middle layers 17B, and a bottom layer 17C comprising a hydrophobic barrier film. In some examples, the upper meltblown polypropylene microfiber material can have a basis weight of 10 to 100 gsm, a fiber size of 1 to 10 microns in diameter, or a combination thereof. Such material is available from Yuhan-Kimberly Corporation, Seoul, Korea.

In some examples, the film barrier layer can be a polyolefin film having a basis weight of 10 to 40 gsm. When a film barrier layer 17C is used in the overall topsheet design, it can include opacifying agents, such as film pigments, that help the film in masking stains along or adjacent to, the pad's side edges 90. In such a fashion, the film layer can serve as a masking element in the pad to limit visualization of a menses insult stain along the central, longitudinal axis L of the pad. The film layer can also serve as a barrier layer to prevent rewet of the topsheet layer user facing surface, as well as to prevent the flow of menses to the side edges of an article. Such film layer can, in some implementations, include apertures, for example, to allow one-way directional transfer of fluid to the multilayer absorbent core.

Referring now to FIG. 4, in an alternative implementation, a topsheet layer 14 can include topographical features 17E which extend out of the overall plane of the topsheet layer 14, and which result from side portions of the topsheet layer rising above the generally planar surface, either as a result of contracted elastic strands, or shrinkable yarns 17F that are laminated to the topsheet layer along the longitudinal axis L, and which shrink upon lamination or alternatively, during contact with menses/moisture. Such strands or yarns, while being shown in two locations in FIG. 4, can be placed in several (typically parallel) longitudinally directed configurations, across the transverse axis of the article. Such a configuration is described for example, in U.S. Patent Application Publication No. 2010/0152690 to Ong.

Referring now to FIG. 5, in an alternative implementation, instead of the central longitudinally directed topsheet material 15 wrapping over the edges of the longitudinally directed side edge topsheet materials 17 and bonded thereto (as seen in FIG. 4, the bond illustrated as 19), the topsheet can alternatively be bonded 19 adjacent the edge. In this fashion, the longitudinally directed side edge topsheet materials 17 can make up the majority of the raised feature on the topsheet layer 14.

Referring again to FIGS. 1 and 2, the feminine hygienic pad 10 comprises a multilayer absorbent core 20, such as those described above, positioned between the topsheet layer 14 and the backsheet layer 12.

The multilayer absorbent core 20 can be shaped and dimensioned so as to be incorporated and function within feminine hygienic pad 10. For example, as illustrated in FIG. 2, in some implementations, the multilayer absorbent core 20 can optionally include longitudinally directed front and rear bulbous portions 21 and indented or cut-out portions 22 along the core side longitudinal edges 41. Such indented or cut-out portions 22 are not structurally necessary, but can be desirable for a compact core design. The multilayer absorbent core 20 can also optionally include an embossing feature 23 for fluid management benefits, or layer stability when wet. In the implementation illustrated, such an embossing feature 23 is not present along the longitudinal and transverse axis central areas of the multilayer absorbent core 20. Such embossing feature may also be present in the center of the absorbent core layer if desired.

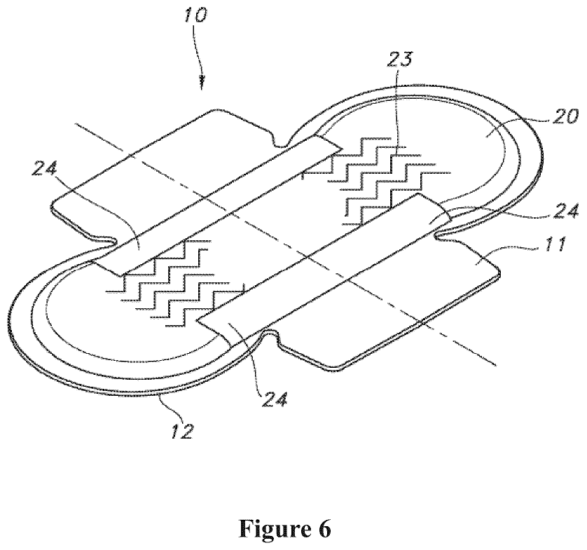

If desired, the multilayer absorbent core can be wrapped in tissue for integrity and/or to facilitate securement of the multilayer absorbent core within the topsheet and backsheet. In one implementation of the multilayer absorbent core, nonwoven side core-edge wraps 24 are initially positioned on the core-facing surface 12A of the backsheet layer 12 for wrapping about the side longitudinally directed edges of the multilayer absorbent core 20, whether or not the core layer includes cutout or indented portions 22. Such nonwoven side core-edge wraps can beconstructed from meltblown microfiber webs as previously described. A wide variety of side core-edge wrap materials is envisaged such as nonwoven sheets, film sheets or laminates thereof. It should be understood that in an alternative implementation (not shown) the side core-edge wraps 24 may be wrapped about a non-indented multilayer absorbent core edge as well. A side perspective view of the partial product can be seen in FIG. 6 with the side core-edge wraps 24 folded over the user facing surface of the multilayer absorbent core 20.

In some implementations, the absorbent article can optionally include additional layers, such as a surge layer. A surge layer can be positioned, for example, between the topsheet layer and the multilayer absorbent core in the depth direction of the absorbent article. A surge layer can be constructed of any woven or nonwoven material that is easily penetrated by body exudates. The surge layer can help to absorb, decelerate, and diffuse surges or gushes of liquid that may be rapidly introduced into the absorbent article. The surge layer can rapidly accept and temporarily hold the liquid prior to releasing the liquid into, for instance, the multilayer absorbent core or any other layer of the absorbent article. Various woven fabrics and nonwoven webs can be used to construct the surge layers. For example, the surge layer can comprise a nonwoven fabric layer composed of a meltblown or spunbond web of polyolefin or polyester filaments. Such nonwoven fabric layers may include conjugate, biconstituent and homopolymer fibers of staple or other lengths and mixtures of such fibers with other types of fibers. The surge layer can also be a bonded card web or an airlaid web composed of natural and/or synthetic fibers. The bonded carded web may, for example, be a powder bonded carded web, an infrared bonded carded web, or a through air bonded carded web. The bonded carded webs can optionally include a mixture or blend of different fibers. In some examples, the surge layer can have a basis weight of less than 100 gsm, and in some implementations, from 10 gsm to 40 gsm.

In some implementations, these articles can exhibit improved intake properties, improved rewet performance, improved saturation and retention capacity, improved desorption, or combinations thereof.

In some implementations, the article can exhibit a rewet value, as determined by the Rewet Test described below, of less than 1.0 g (e.g., 0.95 g or less, 0.90 g or less, 0.85 g or less, 0.80 g or less, 0.75 g or less, 0.70 g or less, 0.65 g or less, or 0.60 g or less). In some implementations, the article can exhibit a rewet value, as determined by the Rewet Test described below, of at least 0.55 g (e.g., at least 0.60 g, at least 0.65 g, at least 0.70 g, at least 0.75 g, at least 0.80 g, at least 0.85 g, at least 0.90 g, at least 0.95 g).

The article can exhibit a rewet value ranging from any of the maximum values described above to any of the maximum values described above. For example, in some implementations, the article can exhibit a rewet value, as determined by the Rewet Test described below, of from 0.55 g to less than 1.0 g (e.g., from 0.55 g to 0.90 g, or from 0.55 g to 0.75 g)

In some implementations, the article can exhibit a retention capacity, as determined by the Retention Capacity Test, of at least 35 g/g (e.g., at least 36 g/g, at least 37 g/g, at least 38 g/g, at least 39 g/g, at least 40 g/g, or at least 41 g/g). In some implementations, the article can exhibit a retention capacity, as determined by the Retention Capacity Test, of 42 g/g or less (e.g., 41 g/g or less, 40 g/g or less, 39 g/g or less, 38 g/g or less, 37 g/g or less, or 36 g/g or less).

The article can exhibit a rewet value ranging from any of the maximum values described above to any of the maximum values described above. For example, in some implementations, the article can exhibit a retention capacity, as determined by the Retention Capacity Test, of from 35 g/g to 42 g/g (e.g., from 35 g/g to 40 g/g, or from 36 g/g to 39 g/g).

### EXAMPLES

The following examples are offered for illustrative purposes and are not intended to limit the scope of the claims in any manner. Those of skill in the art will readily recognize a variety of non-critical parameters which can be changed or modified to yield essentially the same results.

The example multilayer absorbent cores described herein can exhibit improved intake properties, improved rewet performance, improved saturation and retention capacity, improved desorption, or combinations thereof when compared to existing absorbent cores, as demonstrated by the following Examples and Tables.

Sample absorbent articles containing example multilayer absorbent cores described herein were prepared. The properties and performance of these sample articles were then compared to conventional absorbent articles that included alternative multilayer absorbent core architectures. The general method of preparation of the samples was consistent for all examples.

Four absorbent articles were prepared. Example 1 included a multilayer absorbent core having a relatively low basis weight intake layer and a relatively high basis weight absorbent layer, with the ratio of the basis weight of the intake layer to the basis weight of the absorbent layer of from 0.25 to 0.5. Comparative Examples 1-3 include multilayer absorbent cores. However, these multilayer absorbent cores possess relatively lower basis weight absorbent layers (e.g., absorbent layers having a basis weight of less than 200 gsm). Further, the ratio of the basis weight of the intake layer to the basis weight of the absorbent layer in Comparative Examples 1-3 is above 0.5.

The composition of the layers in Example 1 and Comparative Examples 1-3 are detailed in the table below.

| | | Comparative | Comparative | Comparative |
|---|---|---|---|---|
| | Example 1 | Example 1 | Example 2 | Example 3 |
| Topsheet | Hydrophilically | Hydrophilically | Hydrophilically | Hydrophilically |
| | Treated | Treated | Treated | Treated |
| | Embossed | Embossed | Embossed | Embossed |
| | Polypropylene | Polypropylene | Polypropylene | Polypropylene |
| | Spunond | Spunond | Spunond | Spunond |
| | (16-20 gsm) | (16-20 gsm) | (16-20 gsm) | (16-20 gsm) |
| Transfer | Laminate | Thermally | Thermally | Thermally |
| Layer | Including | Bonded Airlaid | Bonded Airlaid | Bonded Airlaid |
| | Bonded Carded | Material | Material | Material |
| | Web, charcoal | Including | Including | Including |
| | printed tissue, | 82%/18% | 82%/18% | 82%/18% |
| | and pulp sheet | Pulp/Binder Fiber | Pulp/Binder Fiber | Pulp/Binder Fiber |
| | (115-140 gsm) | (135-165 gsm; | (135-165 gsm; | (135-165 gsm; |
| | | density of 0.06 | density of 0.06 | density of 0.06 |
| | | g/cc) | g/cc) | g/cc) |
| Intake | Thermally | Thermally | Thermally | Thermally |
| Layer | Bonded Airlaid | Bonded Airlaid | Bonded Airlaid | Bonded Airlaid |
| | Material | Material | Material | Material |
| | Including | Including | Including | Including |
| | 82%/18% | 82%/18% | 82%/18% | 82%/18% |
| | Pulp/Binder | Pulp/Binder | Pulp/Binder | Pulp/Binder |
| | Fiber | Fiber | Fiber | Fiber |
| | (78-102 gsm; | (78-102 gsm; | (88-112 gsm; | (88-112 gsm; |
| | density of 0.06 | density of 0.06 | density of 0.06 | density of 0.06 |
| | g/cc) | g/cc) | g/cc) | g/cc) |
| Absorbent | Airlaid | Airlaid | Airlaid | Airlaid |
| Layer | Including 15% | Including 15% | Including 15% | Including 15% |
| | Superabsorbent | Superabsorbent | Superabsorbent | Superabsorbent |
| | Polymer | Polymer | Polymer | Polymer |
| | Particles, Pulp, | Particles, Pulp, | Particles, Pulp, | Particles, Pulp, |
| | and Top and | and Top and | and Top and | and Top and |
| | Bottom Tissue | Bottom Tissue | Bottom Tissue | Bottom Tissue |
| | Carrier Sheets | Carrier Sheets | Carrier Sheets | Carrier Sheets |
| | (237-283 gsm; | (151-179 gsm; | (151-179 gsm; | (151-179 gsm; |
| | density of 0.34 | density of 0.29 | density of 0.29 | density of 0.29 |
| | g/cc) | g/cc) | g/cc) | g/cc) |
| Back | Embossed | Embossed | Embossed | Embossed |
| Sheet | Polyethylene Film | Polyethylene Film | Polyethylene Film | Polyethylene Film |
| | (21.5-26.3 gsm) | (21.5-26.3 gsm) | (21.5-26.3 gsm) | (21.5-26.3 gsm) |

The absorbent properties of Example 1 and Comparative Examples 1-3 were evaluated using an appropriate menses simulant according to the test procedures described below.

### Saturation Capacity Test

This saturation capacity test was used to determine the amount of menses simulant A (defibrinated swine blood) absorbed (saturation capacity) and retained (retention capacity) by a sample absorbent article. Gram weight and gram per gram results for both saturation and retention capacity were recorded.

Five samples of each article were evaluated, with the results averaged. Each sample was weighed and the weight in grams was recorded as the article's “dry wt.” A non-absorbent was prepared for each sample by peeling away body side liner (topsheet) away from the poly baffle (back sheet) and removing all absorbent core materials. The non-absorbent was then weighed and recorded as the article's “non-absorbent wt.”

Each sample is then placed on a TEFLON coated fiberglass screen having 0.25 inch (0.6 cm) openings (Part #8308, available from Eagle Supply & Plastic, having a place of business in Appleton, Wis., U.S.A.). Each sample was then immersed (body side up) in a bath of menses simulant A for 30 minutes, such that each sample was completely covered by the menses simulant. The bath was placed on a rocker platform (Pro Scientific Pro-512000-00 VSR-50 variable speed rocker with 12″×14″ table, load capacity of 30 lbs, available from Capitol Scientific). The rocker platform was set to a relatively low speed, such that the menses simulant moves freely to both ends of the bath without splashing around. If necessary, additional menses simulant was added during the immersion period to ensure that the samples remain completely submerged.

After 30 minutes, each sample was then removed from the bath, and pooled menses simulant was allowed to drain from the sample. The sample, indicated generally by 308 in FIG. 8, was then placed on test apparatus 300, comprising a vacuum box 302, a TEFLON coated fiberglass screen 304 having 0.25 inch (0.6 cm) openings (Part #8308,available from Eagle Supply & Plastic, having a place of business in Appleton, Wis., U.S.A.) and supported from underneath by a fluorescent light diffuser panel (not shown) and horizontal rods (not shown) to prevent the fiberglass screen 304 from being deformed during the application of the vacuum. A suitable diffuser panel can be obtained as part number 1624K46 from McMaster-Carr Supply Co. (having a place of business in Chicago, Ill., U.S.A.). The diffuser panel can suitably be supported from underneath with three horizontal acrylic rods of ¾″ diameter equally spaced across the width of the vacuum box. The apparatus 300 also included a surgical grade latex cover 306 (Part #8611K16, available from McMaster Carr Supply Company, having a place of business in Chicago, Ill., U.S.A.) sized for overlaying the screen on the vacuum box. The latex was draped over the sample and the entire opening of the vacuum box to form a seal.

More particularly, the absorbent composite sample 308 was placed body side on the screen 304. The screen which previously supported the sample was removed and the sample was allowed to drip for five minutes. The sample 308 was removed from the apparatus and weighed. The weight in grams was recorded as the sample's “wet wt after 5 minute drip.” The sample 308 was then returned to the screen. The latex cover 306 was then placed over the sample 308 and screen 304 (i.e., to generally form a seal over the vacuum box 302) and a vacuum (V) of 0.5 pounds/square inch (34,474 dynes/square cm) was drawn on the vacuum box (and hence the sample) for a period of 5 minutes. The sample 308 was removed from the apparatus and weighed. The weight in grams was recorded as the sample's “wet wt after vacuum.”

The absorbent weight for each sample was determined by the following formula:

Absorbent weight(g)=dry wt−non-absorbentwt

The saturation capacity (drip) for each sample was determined by the following formula:

Saturation Capacity Drip(g/g)=(wet wt after 5 min drip−dry wt)/absorbentwt

The retention capacity (vacuum) for each sample was determined by the following formula:

Retention Capacity Vacuum(g/g)=(wet wt after vacuum−dry wt)/absorbent weight

### Heated Gush Intake Test

The heated gush intake test was used to determine the absorbent performance of example absorbent articles under realistic conditions by adding body temperature and curved placement.

Five samples of each article were evaluated, with the results averaged. Each sample was weighed and the weight in grams was recorded as the article's “dry weight.” The midpoint of each article was located and marked. Each sample was then applied to a flat a strip of white or light-colored cotton underwear material (˜ 1 mm thick, 100% cotton fabric having a basis weight of from 180 gsm to 220 gsm, such as cotton interlock knit white fabric identified by Item #LPIN124920 by Ocean Lanka (Pvt) Ltd, Sri Lanka) cut in the CD direction (3″ CD ×24″ MD).

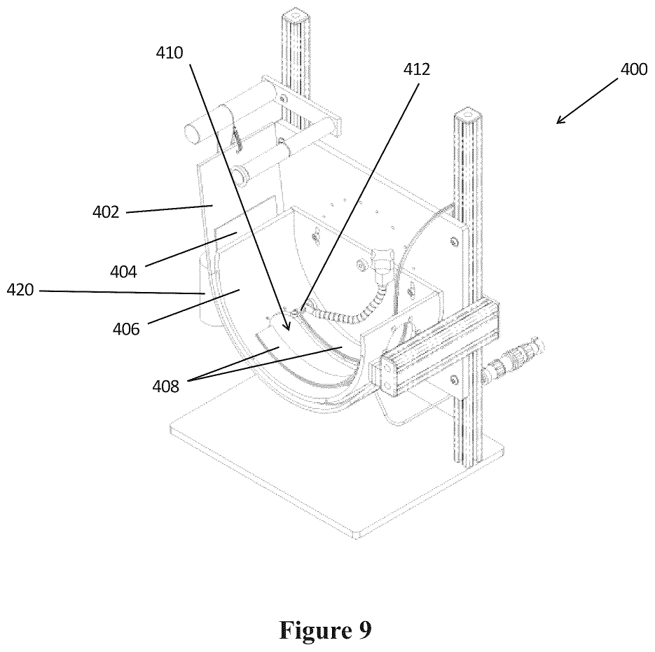

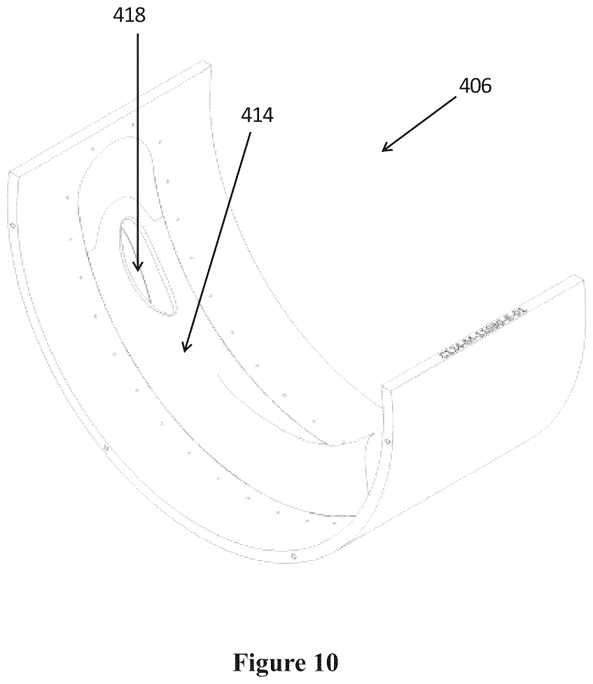

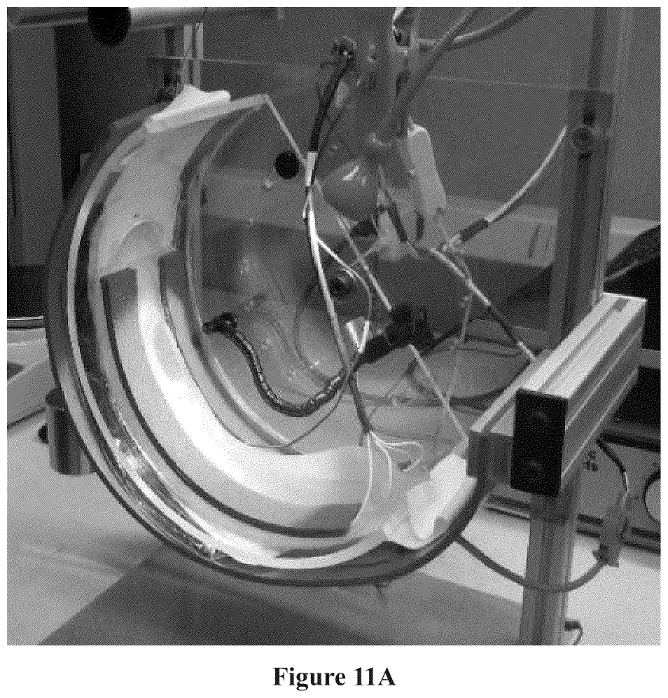

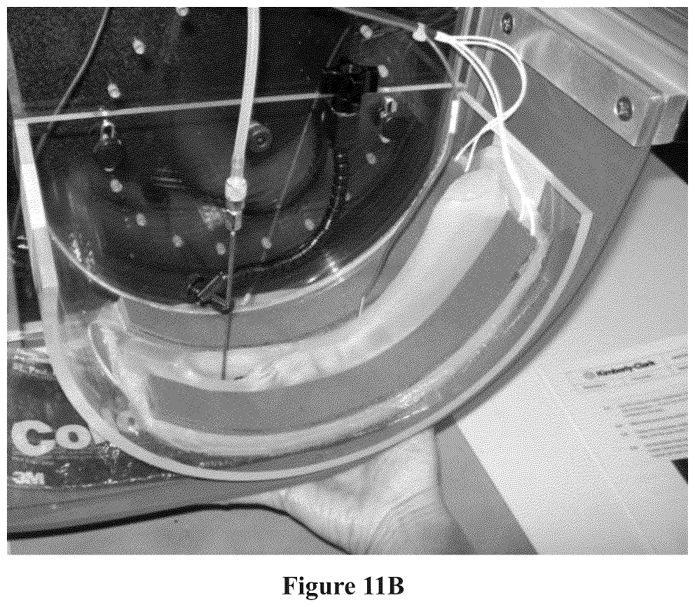

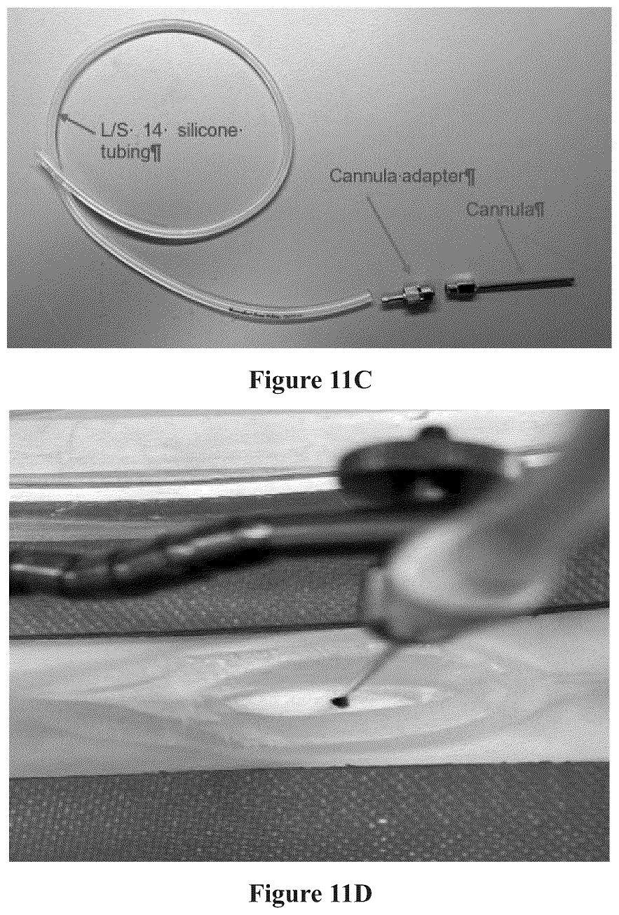

The test apparatus 400, as shown generally in FIGS. 9, 10, and 11A-11D, includes a rubber cradle 402 with a cantilevered weight 420 (500 g) so as to apply upward pressure against a mounting bracket 406. A sample 404 can be affixed to the bottom surface of mounting bracket 406, with the body facing surface of the sample facing away from rubber cradle 402. Mounting bracket 406 is made of clear acrylic and includes a central opening 410 into which an intake mold 414 can be seated, such that the bottom surface of the intake mold (which simulate the anatomy of a wearer) contacts the body facing surface of the sample. Intake mold 414 includes an insult orifice 418 through which menses simulant can be applied to the body facing surface of the article. Insult orifice 418 (39 mm long by ˜10 mm wide) is centered over the midpoint of the article. A pair of heating elements 408 flank central opening 410, allowing the intake mold 414 to be heated to simulate body temperature. The apparatus can further include an adjustable arm 412 that can be articulated to position an interfacing cannula above insult orifice 418, as shown in FIG. 11D, such that menses simulant can be applied through insult orifice 418 to the body facing surface of the article.

Prior to testing, the heating elements were powered and allowed to warm and stabilize for 30-45 minutes to a temperature of 98° F.±2°. A sample was then positioned within the testing apparatus with the marked midpoint located immediately beneath the insult orifice and up against the cradle. The sample was secured to the mounting bracket using double sided tape. A heated 4×10 inch reusable gel cold/hot pack containing ˜170 grams of gel material (commercially available from 3M) was positioned between the sample and the rubber cradle. The cantilevered weight was then engaged by placing the line attached to the weight over a rod. A 60 mL syringe was filled with menses simulant A. #14 silicone tubing is affixed to the tip of the syringe, and the syringe was seated in a syringe pump (PHD2000, Cat #55-4143, S/N: 50697, commercially available from Harvard Apparatus). The syringe pump was programmed to dispense 7 mL of fluid according to the following protocol: (1) 1 mL/minute of menses simulant is dispensed for a period of five minutes; (2) injection is paused for one minute with no fluid dispensed; and (3) a “gush insult” is applied in which 2 mL of fluid is dispensed over a period of five seconds (at a rate of 24 mL/minute). The syringe pump is placed on a rocker platform (Pro Scientific Pro-512000-00 VSR-50 variable speed rocker with 12″×14″ table, load capacity of 30 lbs, available from Capitol Scientific) set to 2. A stainless steel cannula was attached to the #14 silicone tubing via a cannula adaptor (male Luer-Lok nickel plated brass, 1.6 mm (0.0625″) hose barb, Cole Parmer Instrument Company Part #31507-62).

The cannula was seated in the adjustable arm and vertically aligned marked midpoint on the article (˜ 1 cm above the body side of the article). Menses simulant A was dispensed via the syringe pump using the program described above. At the beginning of the gush insult, a stopwatch was started. The progress of the simulant was monitored by visually watching the insult orifice. When the simulant was completely absorbed into the specimen, the stopwatch was stopped. This time was recorded to the nearest second as the Intake Time. If the simulant was not completely absorbed within five minutes, the article was considered to fail.

After five minutes had elapsed, the sample was removed from the apparatus. The stain dimensions on the top (body side) of the specimen, length (MD) and width (CD), and on the bottom (garment/baffle side) of the specimen, length (MD) and width (CD) at the longest and widest points of the stain, respectively, were measured and recorded in mm.

### Rewet Test

This rewet test was used to determine the amount of fluid that will come back to the surface when a load is applied. The amount of fluid that comes back through the surface is called the “rewet” value. The more fluid that comes to the surface, the larger the “rewet” value. Lower rewet values are associated with a dryer material and, thus, a dryer product. In considering rewet, three properties are important: (1) intake, if the material/system does not have good intake then fluid can rewet, (2) ability of absorbent to hold fluid (the more the absorbent holds on to the fluid, the less is available for rewet), and (3) flowback, the more the cover prohibits fluid from coming back through the cover, the lower the rewet.

Rewet tests were performed using an Automatic Blood Strikethrough (AM 1995) Machine which includes a bladder of water on a stand that is driven by an air pump (Omega Engineering, Inc. Model HHP 701) which compresses the bag at 1 psi for 3 minutes.

Two sheets of blotter paper (300 g/m², commercially available under the tradename VERIGOOD® from Ahlstrom) were trimmed to 3.5×12 inches. The sheets of blotter paper were weighed and the weight in grams was recorded as the blotter paper's “dry blotter weight.”

Immediately following completion of the Heated Gush Intake Test described above, a sample was placed on the bladder of the Automatic Blood Strikethrough Machine. The two sheets of pre-weighed trimmed blotter paper were stacked on top of one another and placed on top of the sample, centered over the stain. The lid of the Automatic Blood Strikethrough Machine was closed, and the tester was cycled. During the cycle, the air pump compresses the bladder to create a pressure load of 1.0 psi for 3 minutes on the sample.

Following cycling, the blotter paper and sample were removed from the Automatic Blood Strikethrough Machine. The sample was discarded. Any residual simulant present on the underside of the Automatic Blood Strikethrough Machine cover was then captured by wiping the cover with the blotter paper.

The blotter paper was then weighted, and the weight as the “wet blotter weight.”

The rewet value for each sample was determined by the following formula:

Rewet(g)=(wet blotter weight)−(dry blotter weight)

The properties of Example 1 and Comparative Examples 1-3 are detailed in the tables below.

| | | | | | | Saturation | Retention |
|---|---|---|---|---|---|---|---|
| | | | Pad | Saturation | Retention | Capacity | Capacity |
| | Product | | Weight | Capacity | Capacity | Vacuum | Vacuum |
| | Weight | Thickness | (g) | Drip (g/g) | Drip (g/g) | (g/g) | (g/g) |
| | (g) | (mm) | (std dv) | (std dv) | (std dv) | (std dv) | (std dv) |
| Example 1 | 6.25 | 3.03 | 5.56 | 63.93 | 42.85 | 14.18 | 37.29 |
| | | | (0.06) | (1.779) | (0.525) | (0.291) | (0.498) |
| Comperative | 5.24 | 3.29 | 4.54 | 59.53 | 37.14 | 17.03 | 32.60 |
| Example 1 | | | (0.04) | (1.10) | (0.335) | (0.409) | (0.345) |
| Comparative | 5.68 | 2.96 | 5.07 | 59.59 | 38.73 | 14.93 | 33.66 |
| Example 2 | | | (0.06) | (1.27) | (0.528) | (0.516) | (0.485) |
| Comparative | 5.54 | 3.08 | 4.82 | 56.28 | 36.49 | 15.40 | 31.67 |
| Example 3 | | | (0.05) | (0.0645 | (0.303) | (0.415) | (0.266) |

| | Intake | | | | | |
|---|---|---|---|---|---|---|
| | Gush | | | | | |
| | Intake | Top Stain | Top Stain | Bottom Stain | Bottom Stain | Rewet |
| | Time (sec) | Length (mm) | Width (mm) | Length (mm) | Width (mm) | Rewet (g) |
| | (std dv) | (std dv) | (std dv) | (std dv) | (std dv) | (std dv) |
| Example 1 | 10.2 | 77.2 | 51.7 | 90.0 | 54.8 | 0.655 |
| | (3.27) | (2.64) | (2.34) | (2.76) | (1.83) | (0.039) |
| Comparative | 11.6 | 78.5 | 63.7 | 83.8 | 59.7 | 1.48 |
| Example 1 | (2.23) | (5.01) | (1.97) | (5.49) | (2.16) | (0.29) |
| Comparative | 12.8 | 85.2 | 66.3 | 88.8 | 63.7 | 1.56 |
| Example 2 | (3.01) | (6.31) | (2.66) | (6.91) | (3.39) | (0.08) |
| Comparative | 11.2 | 99.2 | 60.5 | 110 | 59.0 | 1.44 |
| Example 3 | (3.27) | (4.79) | (3.39) | (5.51) | (2.28) | (0.12) |

As shown above, Example 1 exhibits an improved saturation capacity and retention capacity as compared to Comparison Examples 1-3. A decreased gush intake time was observed for Example 1, though this result was not statistically significant. Notably, Example 1 exhibited a significant decrease in rewet as compared to Comparison Examples 1-3. In sum, these results suggest that Example 1 will provide a drier pad for users.

The compositions and methods of the appended claims are not limited in scope by the specific compositions and methods described herein, which are intended as illustrations of a few aspects of the claims. Any compositions and methods that are functionally equivalent are intended to fall within the scope of the claims. Various modifications of the compositions and methods in addition to those shown and described herein are intended to fall within the scope of the appended claims. Further, while only certain representative components, compositions, and method steps disclosed herein are specifically described, other combinations of the components, compositions, and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

### EXAMPLE IMPLEMENTATIONS

Clause 1. A multilayer absorbent core, the core comprising: a transfer layer having a basis weight of from 100 gsm to 175 gsm; an absorbent layer having a basis weight of from 200 gsm to 300 gsm; and an intake layer positioned between and in fluid communication with the transfer layer and the absorbent layer, the intake layer having a basis weight of from 70 gsm to 120 gsm; wherein a ratio of the basis weight of the intake layer to the basis weight of the absorbent layer is from 0.25 to 0.5.

Clause 2. The multilayer absorbent core of clause 1, wherein the ratio of the basis weight of the intake layer to the basis weight of the absorbent layer is from 0.3 to 0.4.

Clause 3. The multilayer absorbent core of any of clauses 1-2, wherein the transfer layer has a basis weight of from 120 gsm to 140 gsm.

Clause 4. The multilayer absorbent core of any of clauses 1-3, wherein the transfer layer comprises a laminated multicomponent nonwoven web.

Clause 5. The multilayer absorbent core of clause 4, wherein the laminated multicomponent nonwoven web comprises cellulosic fibers.

Clause 6. The multilayer absorbent core of any of clauses 1-5, wherein the intake layer has a basis weight of from 80 gsm to 100 gsm.

Clause 7. The multilayer absorbent core of any of clauses 1-6, wherein the intake layer comprises an airlaid nonwoven web.

Clause 8. The multilayer absorbent core of clause 7, wherein the airlaid nonwoven web comprises a blend of cellulosic fibers and binder fibers formed from a synthetic polymer.

Clause 9. The multilayer absorbent core of any of clauses 7-8, wherein the airlaid nonwoven web is thermal bonded.

Clause 10. The multilayer absorbent core of any of clauses 1-9, wherein the absorbent layer has a basis weight of from 250 gsm to 270 gsm.

Clause 11. The multilayer absorbent core of any of clauses 1-10, wherein the absorbent layer comprises an airlaid nonwoven web.

Clause 12. The multilayer absorbent core of any of clauses 1-11, wherein the absorbent layer comprises cellulosic fibers.

Clause 13. The multilayer absorbent core of any of clauses 1-12, wherein the absorbent layer comprises from 5% by weight to 50% by weight of a superabsorbent polymer composition.

Clause 14. The multilayer absorbent core of any of clauses 1-13, wherein the absorbent layer is more hydrophilic than the transfer layer.

Clause 15. A personal care absorbent article for receiving a body exudate, the article comprising a liquid-permeable topsheet layer, a liquid-impermeable backsheet layer, and a multilayer absorbent core defined by any of clauses 1-14 positioned between the topsheet layer and the backsheet layer.

Clause 16. The article of clause 15, wherein the article exhibits a rewet value of less than 1.0 g, as determined by a Rewet Test, such as a rewet value of from 0.55 g to 0.90 g, or from 0.55 g to 0.75 g.

Clause 17. The article of any of clauses 15-16, wherein the article exhibits a retention capacity of at least 35 g/g, as determined by a Retention Capacity Test, such as a retention capacity of from 35 g/g to 42 g/g.

Clause 18. The article of any of clauses 15-17, wherein the article is a diaper, a training pant, a youth pant, a swim pant, a feminine hygiene product, an incontinence product, a medical garment, a surgical pad, or a bandage.

Clause 19. The article of clause 18, wherein the feminine hygiene product is a menstrual pad or menstrual pant.

1. A multilayer absorbent core, the core comprising:
a transfer layer having a basis weight of from 100 gsm to 175 gsm;
an absorbent layer having a basis weight of from 200 gsm to 300 gsm; and
an intake layer positioned between and in fluid communication with the transfer layer and the absorbent layer, the intake layer having a basis weight of from 70 gsm to 120 gsm;
wherein a ratio of the basis weight of the intake layer to the basis weight of the absorbent layer is from 0.25 to 0.5.

2. The multilayer absorbent core of claim 1, wherein the ratio of the basis weight of the intake layer to the basis weight of the absorbent layer is from 0.3 to 0.4.

3. The multilayer absorbent core of claim 1, wherein the transfer layer has a basis weight of from 120 gsm to 140 gsm.

4. The multilayer absorbent core of claim 1, wherein the transfer layer comprises a laminated multicomponent nonwoven web.

5. The multilayer absorbent core of claim 4, wherein the laminated multicomponent nonwoven web comprises cellulosic fibers.

6. The multilayer absorbent core of claim 1, wherein the intake layer has a basis weight of from 80 gsm to 100 gsm.

7. The multilayer absorbent core of claim 1, wherein the intake layer comprises an airlaid nonwoven web.

8. The multilayer absorbent core of claim 7, wherein the airlaid nonwoven web comprises a blend of cellulosic fibers and binder fibers formed from a synthetic polymer.

9. The multilayer absorbent core of claim 7, wherein the airlaid nonwoven web is thermal bonded.

10. The multilayer absorbent core of claim 1, wherein the absorbent layer has a basis weight of from 250 gsm to 270 gsm.

11. The multilayer absorbent core of claim 1, wherein the absorbent layer comprises an airlaid nonwoven web.

12. The multilayer absorbent core of claim 1, wherein the absorbent layer comprises cellulosic fibers.

13. The multilayer absorbent core of claim 1, wherein the absorbent layer comprises from 5% by weight to 50% by weight of a superabsorbent polymer composition.

14. The multilayer absorbent core of claim 1, wherein the absorbent layer is more hydrophilic than the transfer layer.

15. A personal care absorbent article for receiving a body exudate, the article comprising a liquid-permeable topsheet layer, a liquid-impermeable backsheet layer, and a multilayer absorbent core defined by claim 1 positioned between the topsheet layer and the backsheet layer.

16. The article of claim 15, wherein the article exhibits a rewet value of less than 1.0 g, as determined by a Rewet Test, such as a rewet value of from 0.55 g to 0.90 g, or from 0.55 g to 0.75 g.

17. The article of claim 15, wherein the article exhibits a retention capacity of at least 35 g/g, as determined by a Retention Capacity Test, such as a retention capacity of from 35 g/g to 42 g/g.

18. The article of claim 15, wherein the article is a diaper, a training pant, a youth pant, a swim pant, a feminine hygiene product, an incontinence product, a medical garment, a surgical pad, or a bandage.

19. The article of claim 18, wherein the feminine hygiene product is a menstrual pad or menstrual pant.
